(19) United States
(12) Patent Application Publication
(10) Pub. No.: US 2024/0009212 A1
(43) Pub. Date: Jan. 11, 2024

(54) PHOSPHAPLATIN COMPOUNDS AS THERAPEUTIC AGENTS SELECTIVELY TARGETING HIGHLY GLYCOLYTIC TUMOR CELLS AND METHODS THEREOF

(71) Applicant: Promontory Therapeutics Inc., New York, NY (US)

(72) Inventors: Tyler D. Ames, Brooklyn, NY (US); Alberto Anel, Zaragoza (ES)

(73) Assignee: Promontory Therapeutics Inc., New York, NY (US)

(21) Appl. No.: 18/249,783

(22) PCT Filed: Oct. 20, 2021
(86) PCT No.: PCT/US21/55907

§ 371 Date: Apr. 20, 2023

Related U.S. Application Data
(60) Provisional application No. 63/094,048, filed on Oct. 20, 2020.

(51) Int. Cl. A61K 31/6615; A61K 45/06; A61P 35/00; G01N 33/574

(52) U.S. Cl. CPC A61K 31/6615; A61K 45/06; A61P 35/00; G01N 33/57484; G01N 2333/4703

(57) ABSTRACT

A cellular model with a highly glycolytic phenotype (L929dt cells) for study of phosphaplatin-based anticancer agents, in particular (R,R)-1,2-cyclohexanediamine-(pyrophosphato) platinum(II) (or “PT-112”), is disclosed. The expression of HIF-1α as a biomarker of glycolytic cells sensitive to PT-112, clinical applications of the biomarker, and methods thereof for diagnosis and treatment of patients with cancers are disclosed.

### CROSS-REFERENCE TO RELATED APPLICATION

This application claims priority under 35 U.S.C. § 119(e) to U.S. Provisional Patent Application No. 63/094,048, filed on Oct. 20, 2020, the disclosure of which is incorporated herein by reference in its entirety.

### FIELD OF THE DISCLOSURE

The present disclosure relates to a biomarker for identifying glycolytic tumor cells susceptible to treatment by phosphaplatin anticancer agents and application of the biomarker to methods of target treatment of various cancers.

### BACKGROUND OF THE DISCLOSURE

Among the many modes of drug resistance within the context of cancer therapeutics, hypoxia has long been known to play an important and particularly challenging role, especially in advanced, metastatic cancer (Jing, X., et al. Mol Cancer 18, 157 (2019)). It was long thought that this might relate physiologically to the lack of oxygen in the center of a large, growing tumor mass, leading to changes in cancer metabolism (toward a glycolytic phenotype). With recent understanding of cancer on the basis of molecular and signaling pathway research, and with the concept of the tumor-microenvironment (TME), it has since been shown that hypoxia can affect cancer resistance to therapy across a wide range of pathways, with the potential to lead to acquired resistance to chemotherapy, radiation therapy and immuno-therapy, and associated poor prognosis in cancer patients. Furthermore, this resistance has been demonstrated in relation to the inhibition of DNA damage by DNA damaging/binding agents (C. Wigerup et al., Pharmacology & Therapeutics 164 (2016) 152-169), which describes the canonical understanding of the mechanism of cancer cell death by platinum-containing chemotherapies.

In 2019, the Nobel Prize in Physiology or Medicine was awarded for work in characterizing how “animal cells undergo fundamental shifts in gene expression when there are changes in the oxygen levels around them.” (see: https://www.nobelprize.org/prizes/medicine/2019/advanced-information/) (last accessed on Oct. 18, 2021). In part, this work involved Gregg Semenza's identification of the so-called Hypoxia Inducible Factor, including the molecular target HIF-1α now considered a relevant factor in cancer cell signaling, and thus in therapeutic intervention. The literature built on these discoveries to characterize HIF-1 and HIF-2 as potential therapeutic targets in oncology (C. Wigerup et al.). In 2020, the first clinical proof of concept data was reported in relation to single-targeted therapeutic intervention directed to HIF2-α (Srinivasan, R. et al., Annals of Oncology (2020) 31 (suppl_4)).

The role of hypoxia is therefore established both as a factor involved in drug resistance in cancer patients, representing a challenge in patient care, and as a validated target for therapeutic intervention, representing an opportunity for improvement in care. Given the role of hypoxic factors in tumor resistance to chemotherapies, such as platinum-containing chemotherapies, it would therefore be unexpected to discover that a platinum-containing agent might have selectivity in inducing cell death of glycolytic cells or those with high expression of hypoxia inducible factor(s).

Platinum-based therapy continues to be at the backbone of pharmacological intervention in solid tumor therapy (Hellmannm, M., et al. (2016) Ann Oncol, 27:1829-1835). Notably, platinum salts, such as cisplatin and carboplatin are showing to be the best companions for combination therapy with immunotherapy mediated by checkpoint inhibitors (Paz-Ares, L., et al. (2018) New Eng J Med, 379:2040-2051; Horn, L., et al. (2018) New Eng J Med, 379:2220-2229). Moreover, cis and carboplatin-based therapies have limitations in terms of toxicity, reducing their feasibility for sub-chronic therapy. For instance, it is considered that up to 50% of urothelial cancer patients are not eligible for platin-based therapies due to co-morbidities (De Santis, M., (2013) Eur Oncol Haematol Suppl, DOI: 10.17925/EOH.2013.09.S1.13). These platinum salts, which are essentially DNA binders, are subjected to acquired cancer cell resistance through acute activation of DNA repair pathways (Kelland, L., (2007) Nature Rev Cancer, 7:573-584). Therefore, the identification of a new generation of Pt-containing chemical entities that could exert their anti-cancer activity through non-DNA-mediated mechanisms is a major priority in drug development.

In this respect, the R,R-1,2 cyclohexanediamine-pyrosphosphato-platinium (II) (PT-112) is the result of a major effort in the medical chemistry field to construct a stable pyrophosphate containing conjugate with a diaminocyclohexane-Pt ring (Bose, R., et al. (2008) Proc. Natl. Acad. Sci. USA, 105:18314-18319). The primary objective of this drug discovery program was: i) to propose a new class of anticancer agents active through a non-DNA binding mediated cancer cell death; ii) to propose a stable chemical entity with lack of acute chemical degradation to multiple metabolites and minimal protein binding affinity; and iii) to propose an anticancer agent lacking acute renal toxicities and acute neurotoxicity, hypothesis confirmed in in vivo validated experimental models.

PT-112 is a novel stable pyrophosphate containing conjugate with a link to a diaminocyclohexane-platinum ring, with clinical activity in advanced pre-treated solid tumors including non-small cell lung cancer, small cell lung cancer, thymoma, and castration resistant prostate cancer (CRPC) (Karp et al., Annals of Oncology (2018) 29 (suppl_8). The molecular model of PT-112 target disruption in cancer cells is under investigation, but previous observations indicate its marked induction of immunogenic cell death, a mode of regulated cell death that promotes the adaptive immune response (Yamazaki, et al, OncoImmunology 2020 February 11; 9(1):1721810). Observations also suggest that its cancer cell selectivity could be related to the metabolic status of tumor cells. Of note, and contrary to other more classic chemotherapeutics, PT-112 lacks major DNA binding. There is a need for a biomarker for identifying tumor cells susceptible to treatment by phosphaplatin anticancer agents and application of the biomarker to methods of target treatment of various cancers in future clinical applications.

### SUMMARY OF THE DISCLOSURE

This disclosure addresses the above-mentioned need by providing methods for diagnosing a cancer patient for treatment with a phosphaplatin compound. The disclosure is based on a surprising discovery of the extended study of PT-112, in particular mechanistic study using a novel cellular model.

In one aspect, the present disclosure relates to use of HIF-1a expression in glycolytic cells as a biomarker in determining potential effectiveness of phosphaplatin compounds in the treatment of a cancer patient.

In one aspect, the present disclosure relates to a method of diagnosing a cancer patient for treatment with a phosphaplatin compound, comprising measuring expression of HIF-1α in glycolytic cells of the cancer patient, wherein an expression of HIF-1α at a defined level indicates that the cancer patient can potentially be treated with the phosphaplatin compound effectively.

In one aspect, the present disclosure relates to a method of treating a cancer tumor, comprising the steps of
(a) measuring the expression level of HIF-1α in glycolytic cells of the patient; and
(b) if the expression level of HIF-lu in the glycolytic cells obtained in the step (a) is at or above a defined level, administering to the patient a therapeutically effective amount of a phosphaplatin compound.

In one aspect, the present disclosure relates to a method of inhibiting proliferation of tumor cells characterized by a highly glycolytic phenotype, comprising contacting the cells with a phosphaplatin compound.

In one embodiment, the phosphaplatin compound has a structure of formula I or II:

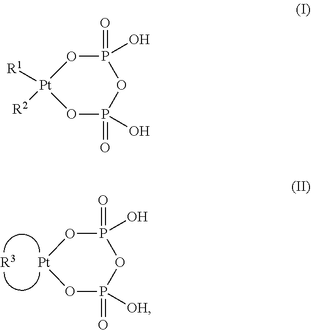

or a pharmaceutically acceptable salt thereof, wherein R¹ and R² are each independently selected from NH₃, substituted or unsubstituted aliphatic amines, and substituted or unsubstituted aromatic amines; and wherein R³ is selected from substituted or unsubstituted aliphatic diamines, and substituted or unsubstituted aromatic diamines.

In a particular preferred embodiment, the phosphaplatin compound is (R,R)-1,2-cyclohexanediamine-(pyrophosphato)platinum(II) (or “PT-112”), or a pharmaceutically acceptable salt thereof.

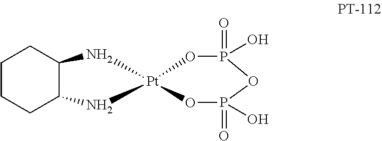

The cancers or tumors that can be treated according to the present disclosure include, but are not limited to, gynecological cancers, genitourinary cancers, lung cancers, head-and-neck cancers, skin cancers, gastrointestinal cancers, breast cancers, bone and chondroital cancers, soft tissue sarcomas, thymic epithelial tumors, and hematological cancers.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description, drawings, examples, and claims.

### BRIEF DESCRIPTION OF THE DRAWINGS

FIGS. 1A and 1B (collectively “FIG. 1”) illustrate the cell growth analysis after treatment with increasing concentrations of PT-112 (FIG. TA) and Cisplatin (FIG. 1B) separately, incubated for 24-72 h. As indicated, FIGS. 1A and 1B show results obtained with PT-112 and cisplatin incubations, respectively. Results were expressed as the percentage of relative growth compared to control, untreated cells±SD of at least two (2) independent experiments made in duplicate.

FIGS. 2A and 2B (collectively “FIG. 2”) illustrate cytotoxic assays after treatment with PT-112 or Cisplatin. Parental cells L929, L929dt and cybrids cells were incubated with 10 μM of PT-112 or cisplatin for 24, 48 and 72 h and then simultaneously stained with annexin-V-FITC and 7-AAD and analyzed by flow cytometry. The dot-plots in FIG. 2A show the cell population evolution upon PT-112 treatment. FIG. 2B The graph-bars in FIG. 2B correspond to data representation indicating the percentage of the single or double-labelled cell populations. Results are shown as median±SD of at least two (2) independent experiments made in duplicate.

FIG. 3 shows the analysis of mitochondrial membrane potential (ΔΨₘ) upon treatment with PT-112 at different incubation times. Cells (3×10⁴) were incubated with 10 μM of PT-112 for 24, 36, 48 and 72 h at 37° C. Changes in ΔΨₘ was determined by staining with DiOC₆ and analyzed by flow cytometry. As shown in the legend, dotted-lines correspond to MFI of non-treated cells and grey-tinted lines the MFI of treated cells.

FIGS. 4A and 4B (collectively “FIG. 4”) illustrate caspase-3 activation by PT-112 and effect of caspase and necrostatin-1 inhibitors. FIG. 4A illustrates the levels of caspase-3 activation upon treatment with PT-112. The numbers in each box represent the percentage of cleaved caspase-3 compared to non-treated cells. FIG. 4B shows cytotoxicity analysis of PT-112 combined with Z-VAD-fmk and necrostatin-1 inhibitors. Results are shown as median SD of three independent experiments made in duplicate.

FIGS. 5A, 5B and 5C (collectively “FIG. 5”) illustrate the analysis of total and specific mitochondrial ROS production upon treatment with PT-112 at different incubation times. (A) Cells (3×10⁴) were incubated with 10 μM of PT-112 for 24, 36, 48 and 72 h at 37° C. Total ROS production was determined by staining with 2HE and flow cytometry. (B) Graphical representation of data obtained in FIG. 5A. It shows as medium fluorescence intensity (MFI) of treated cells compared to non-treated cells. (C) Specific mitochondrial ROS production after incubation with 10 μM of PT-112. Cells were stained with a mitochondrial superoxide indicator MitoSOX™ for 15 minutes at 37° C., in darkness. The fluorescence intensity of treated cells compared to control cells was determined by flow cytometry. As shown in the legend, dotted-lines corresponds to MFI of non-treated cells and grey-tinted lines the MFI of treated cells.

FIG. 6 illustrates the effect of antioxidant glutathione (GSH) on PT-112 induced-cell death upon 72 h. L929dt and L929^{dt} cybrid cells were pretreated with 5 mM GSH for 1 h and subsequently incubated with 10 μM of PT-112 for 72 h. Bars represented as “Pre-GSH+PT-112” corresponds to data obtained from cells treated with a mixture of 10 μM of PT-112 and 5 mM GSH, both drugs previously incubated 1 h in absence of cells. Cell death was evaluated using annexin-V-FITC and 7-AAD stanning by flow cytometry. Results are shown as median SD of at least 3 independent experiments made in duplicate. *** p≤0.0001.

FIGS. 7A, 7B and 7C (collectively “FIG. 7”) illustrate partial inhibition of PT-112-induced mtROS generation and cell death in L929dt cells by the mtROS scavenger MitoTempo. (A) Cell death was evaluated by flow cytometry using annexin-V-FITC and 7-AAD staining. (B) mtROS levels were measured using MitoSOX™ staining as described previously. (C) Antimycin A, a mtROS inductor, was used as a positive control. Results are shown as median±SD of at least 2 independent experiments made in duplicate. * p≤0.05.

FIG. 8 illustrates cell growth analysis after treatment of L929-ρ⁰ cells with PT-112 and Cisplatin. Cells were treated with increasing concentrations of PT-112 and cisplatin separately, incubated for 24-72 h and relative growth was measured by MTT assay method. Results correspond to percentage of growth inhibition with respect to untreated control cells. Results are shown as median±SD of at least 2 independent experiments made in duplicate. * p<0.05, ** p≤0.01.

FIGS. 9A and 9B (collectively “FIG. 9”) illustrate that PT-112 induces mitochondrial membrane depolarization in LNCap-C4 prostate cancer cell line as measured by flow cytometry. FIG. 9A shows that PT-112 induces mitochondrial membrane depolarization concurrently with mtROS. In FIG. 9B, flow cytometry shows loss in mitochondrial membrane potential correlates over time with cell death.

FIGS. 10A, 10B and 10C (collectively “FIG. 10”) illustrate that PT-112 induces the initiation of autophagy. FIG. 10A shows the analysis of autophagosome formation. Cells were incubated with 10 μM of PT-112 for 48-72 h. The autophagosomes formation was analyzed by flow cytometry using Cyto-ID® method. FIG. 10B is a graphical representation of data obtained with in Cyto-ID® analysis. It shows as medium fluorescence intensity (MFI) of treated cells compared to non-treated cells. FIG. 10C shows expression levels of p62 and LC3BI/II upon PT-112 treatment. Tubulins are used as a control of protein loaded. Cytotoxic effect of combining PT-112 with rapamycin in Warburg-dependent cell lines. Cells (3×10⁴) were incubated for 48 h with PT-112 alone or in combination with rapamycin. Percentage of cell death was analyzed by flow cytometry using annexin-V-FITC and 7-AAD staining. Results are shown as median±SD of three independent experiments made in duplicate.

FIG. 11 shows cell morphology after PT-112 treatment. Phase-contrast micrographs of cells treated or not (CTRL) with 10 μM PT-112 for 72 h are shown.

FIG. 12 shows effects of PT-112 on Rab5. The indicated cell lines were treated or not (CTRL) with 10 μM PT-112 for the time indicated, cell extracts obtained, cell proteins separated by SDS-PAGE and immunboloted with a specific anti-Rab5 antibody. An anti-b-actin immunoblot was performed on the same membranes as loading control.

FIG. 13 shows an analysis of HIF-lu expression levels in the presence or absence of PT-112. Cells were incubated with 10 μM of PT-112 for 72 h. Cell lysates were resolved in a SDS-PAGE 6% polyacrylamide gel, proteins were transferred on nitrocellulose membrane and incubated with a specific antibody against HIF-1a. R-Actin was used as a control of protein loaded. Annexed table shows the percentage of protein expression in basal conditions with respect to parental cell L929.

### DETAILED DESCRIPTION OF THE DISCLOSURE

Phosphaplatins have been identified as a class of compounds useful for the treatment of cancers resistant to cisplatin and carboplatin. See, e.g., U.S. Pat. Nos. 8,034,964; 8,445,710; and 8,653,132. In particular, R,R-1,2-cyclohexanediamine-pyrophosphato-platinum (II) (PT-112) has entered clinical studies in the treatment of various cancers, e.g., non-small cell lung cancer (NSCLC), urothelial carcinoma (UC), squamous cell carcinoma of the head and neck (SCCHN), metastatic breast cancer (mBC), castration-resistant prostate cancer (CRPC), and multiple myeloma. See, e.g., U.S. Pat. Nos. 9,688,709; 10,385,083; and 10,364,264; and WO 2018/129257. Synthetic and purification methods of PT-112 and formulations for parenteral administration have been reported. See, e.g., U.S. Pat. Nos. 8,846,964; 8,859,796; and WO 2017/176880. All of the relevant patent references cited herein concerning preparation of PT-112 and analogs, and pharmaceutical compositions and medical uses thereof are incorporated herein by reference as if they were set forth fully in this disclosure.

The inventors have previously established a cellular model with an extreme glycolytic phenotype (L929dt cells) vs. its parental OXPHOS-competent cell line (L929 cells), together with mitochondrial cybrids that reproduced both phenotypes (L929^{dt} and dt^{L929} cells, respectively). This cellular system could be used to explore metabolic dependence for the PT-112's molecular pharmacodynamics profile, since glycolytic tumor cells presenting mutations in mtDNA (L929dt and L929^{dt} cybrid cells) are especially sensitive to cell death induced by PT-112 while tumor cells with an intact Oxphos pathway (L929 and dt^{L929} cybrid cells) are less sensitive to PT-112. As a control, all cells are sensitive to the classical Pt-containing drug cisplatin. Contrary to cisplatin, the type of cell death induced by PT-112 does not follow the classical apoptotic pathway.

In addition, although PT-112 induces caspase-3 activation at the same time as cell death, the general caspase inhibitor Z-VAD-fmk does not inhibit PT-112-induced cell death, alone or in combination with the necroptosis inhibitor necrostatin-1. PT-112 induces a massive mitochondrial reactive oxygen species (ROS) accumulation only in the most sensitive, glycolytic cells, together with mitochondria hyperpolarization. PT-112 induces the initiation of autophagy in all cell lines, but it seems that the autophagy process is not completed, since p62 is not degraded. PT-112 also affected Rab5 prenylation and dimerization status, indicating that it is disrupting the mevalonate pathway. Mevalonate pathway inhibition blocks production of ubiquinone which then induces mitochondrial oxidative stress consistent with high levels of ROS accumulation. Finally, the expression of HIF-1α is much higher in glycolytic cells especially sensitive to PT-112 than in cells with an intact oxphos pathway.

This disclosure addresses the above-mentioned need by providing methods for diagnosing a cancer patient for treatment with a phosphaplatin compound. The disclosure is based on a surprising discovery of the extended study of PT-112, in particular mechanistic study using a novel cellular model.

In one aspect, the present disclosure relates to use of HIF-1α expression in glycolytic cells as a biomarker in determining potential effectiveness of phosphaplatin compounds in the treatment of a cancer patient.

In one aspect, the present disclosure relates to a method of diagnosing a cancer patient for treatment with a phosphaplatin compound, comprising measuring expression of HIF-1α in glycolytic cells of the cancer patient, wherein an expression of HIF-1α at a defined level indicates that the cancer patient can potentially be treated with the phosphaplatin compound effectively.

In one aspect, the present disclosure relates to a method of treating a cancer tumor, comprising the steps of
(a) measuring the expression level of HIF-1α in glycolytic cells of the patient; and
(b) if the expression level of HIF-1α in the glycolytic cells obtained in the step (a) is at or above a defined level, administering to the patient a therapeutically effective amount of a phosphaplatin compound.

In some embodiments, the defined level of HIF-1α is 1.2 times, 1.5 times, 2.0 times, 2.5 times, 3.0 times, 3.5 times, 4.0 times, 5.0 times, or 6.0 times the expression level of HIF-1α in parental cells.

In some preferred embodiments, the defined expression level of HIF-1α is 2.0 times the expression level of HIF-1α in parental cells.

In some preferred embodiments, the defined expression level of HIF-1α is 3.0 times the expression level of HIF-1α in parental cells.

In some preferred embodiments, the defined expression level of HIF-1α is 4.0 times the expression level of HIF-1α in parental cells.

In some preferred embodiments, the defined expression level of HIF-1α is 5.0 times the expression level of HIF-1α in parental cells.

In some preferred embodiments, the defined expression level of HIF-1α is 6.0 times the expression level of HIF-1α in parental cells.

In one aspect, the present disclosure relates to a method of inhibiting proliferation of tumor cells characterized by a highly glycolytic phenotype, comprising contacting the cells with a phosphaplatin compound.

In some embodiments, the highly glycolytic phenotype is characterized by an expression level of HIF-1μ in glycolytic cells that is at least 1.2 times, at least 1.5 times, at least 2.0 times, at least 2.5 times, at least 3.0 times, at least 4.0 times, at least 4.5 times, at least 5.0 times, at least 5.5 times, or at least 6.0 times the expression level of HIF-1α in parental cells.

In some preferred embodiments, the expression level of HIF-1α in glycolytic cells that is at least 2.0 times the expression level of HIF-1α in parental cells.

In some preferred embodiments, the expression level of HIF-1α in glycolytic cells that is at least 3.0 times the expression level of HIF-1α in parental cells.

In some preferred embodiments, the expression level of HIF-1α in glycolytic cells that is at least 4.0 times the expression level of HIF-1α in parental cells.

In some preferred embodiments, the expression level of HIF-1α in glycolytic cells that is at least 5.0 times the expression level of HIF-1α in parental cells.

In some preferred embodiments, the expression level of HIF-1α in glycolytic cells that is at least 6.0 times the expression level of HIF-1α in parental cells.

In one embodiment, the phosphaplatin compound has a structure of formula I or II:

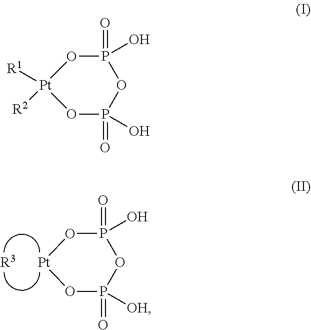

or a pharmaceutically acceptable salt thereof, wherein R¹ and R² are each independently selected from NH₃, substituted or unsubstituted aliphatic amines, and substituted or unsubstituted aromatic amines; and wherein R³ is selected from substituted or unsubstituted aliphatic diamines, and substituted or unsubstituted aromatic diamines.

In one embodiment, in the phosphaplatin compound having a structure of formula I or II, R¹ and R² are each independently selected from NH₃, methyl amine, ethyl amine, propyl amine, isopropyl amine, butyl amine, cyclohexane amine, aniline, pyridine, and substituted pyridine; and R³ is selected from 1,2-ethylenediamine and cyclohexane-1,2-diamine.

In one embodiment, the phosphaplatin compound is selected from the group consisting of:

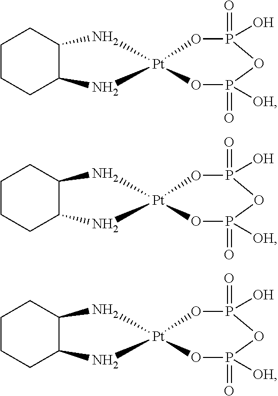

or pharmaceutically acceptable salts, and mixtures thereof.

In one embodiment, the phosphaplatin compound is (R,R)-1,2-cyclohexanediamine-(pyrophosphato)platinum(II) (or “PT-112”), or a pharmaceutically acceptable salt thereof.

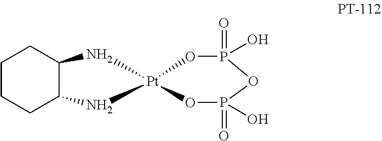

In one embodiment, the cancer or tumor is selected from the group consisting of gynecological cancers, genitourinary cancers, lung cancers, head-and-neck cancers, skin cancers, gastrointestinal cancers, breast cancers, bone and chondroital cancers, soft tissue sarcomas, thymic epithelial tumors, and hematological cancers.

In one embodiment, the bone or blood cancer is selected from the group consisting of osteosarcoma, chondrosarcoma, Ewing tumor, malignant fibrous histiocytoma (MFH), fibrosarcoma, giant cell tumor, chordoma, spindle cell sarcomas, multiple myeloma, non-Hodgkin lymphoma, Hodgkin lymphoma, leukemia, childhood acute myelogenous leukemia (AML), chronic myelomonocytic leukaemia (CMML), hairy cell leukaemia, juvenile myelomonocytic leukaemia (JMML), myelodysplastic syndromes, myelofibrosis, myeloproliferative neoplasms, polycythaemia vera, and thrombocythaemia.

In one embodiment, the bone or blood cancer is selected from the group consisting of osteosarcoma, chondrosarcoma, Ewing tumor, malignant fibrous histiocytoma (MFH), fibrosarcoma, giant cell tumor, chordoma, spindle cell sarcomas, multiple myeloma, non-Hodgkin lymphoma, Hodgkin lymphoma, leukemia.

In one embodiment, the method of treatment is in conjunction with administering to the subject a second anti-cancer agent.

In one embodiment, the second anti-cancer agent is selected from the group consisting of alkylating agents, glucocorticoids, immunomodulatory drugs (IMiDs), proteasome inhibitors, and checkpoint inhibitors.

In one embodiment, the immunomodulatory drugs (IMiDs) are selected from the following group: 6Mercaptopurine, 6MP, Alferon N, anakinra, Arcalyst, Avonex, Avostartgrip, Bafiertam, Berinert, Betaseron, BG-12, C1 esterase inhibitor recombinant, C1 inhibitor human, Cinryze, Copaxone, dimethyl fumarate, diroximel fumarate, ecallantide, emapalumab, emapalumab-lzsg, Extavia, fingolimod, Firazyr, Gamifant, Gilenya, glatiramer, Glatopa, Haegarda, icatibant, Infergen, interferon alfa n3, interferon alfacon 1, interferon beta 1a, interferon beta 1b, Kalbitor, Kineret, mercaptopurine, monomethyl fumarate, peginterferon beta-1a, Plegridy, Purinethol, Purixan, Rebif, Rebidose, remestemcel-L, rilonacept, ropeginterferon alfa 2b, Ruconest, Ryoncil, siltuximab, sutimlimab, Sylvant, Tecfidera or Vumerity.

In one embodiment, the proteasome inhibitors may include, by way of example only, Velcade (bortezomib), Kyprolis (carfilzomib), and Ninlaro (ixazomib).

In one embodiment, the checkpoint inhibitor is selected from the group consisting of PD-1 inhibitors, PD-L1 inhibitors, B7-1/B7-2 inhibitors, CTLA-4 inhibitors, and combinations thereof.

In one embodiment, the PD-1 inhibitor may include, by way of example, Opdivo (nivolumab), Keytruda (pembrolizumab) or Libtayo (cemiplimab).

In one embodiment, the PD-L1 inhibitor may include, by way of example, Tecentriq (atezolizumab), Bavencio (avelumab), or Imfinzi (durvalumab).

In another aspect, the present disclosure provides a method of treating a cancer in a subject diagnosed to be treatable with a phosphaplatin compound of formula (I) or (II) disclosed herein, especially PT-112, the method comprising administering to the subject a therapeutically effective amount of a sterile liquid formulation comprising a phosphaplatin compound (e.g., PT-112) in an aqueous buffer solution, as disclosed in WO 2017/176880, which is incorporated by reference as if it were fully set forth herein as the part of the disclosure.

In some embodiments, the liquid formulation of phosphaplatin compound (e.g., PT-112) has a pH in the range of about 7 to about 9. In some embodiments, the pH is about 7.0 to about 8.0.

In some embodiments, the liquid formulation of phosphaplatin compound (e.g., PT-112) is a ready-to-use liquid formulation suitable for parenteral administration.

In some embodiments, the liquid formulation of phosphaplatin compound (e.g., PT-112) has a concentration of the phosphaplatin compound about 20 mg/mL or less.

In some embodiments, the liquid formulation of phosphaplatin compound (e.g., PT-112) has a concentration of the phosphaplatin compound between about 1 and about 10 mg/mL.

In some embodiments, the liquid formulation of phosphaplatin compound (e.g., PT-112) has a concentration of the phosphaplatin compound between about 1 and about 6 mg/mL.

In some embodiments, the liquid formulation of phosphaplatin compound (e.g., PT-112) has a concentration of the phosphaplatin compound about 5 mg/mL.

In some embodiments, the buffer solution of liquid formulation comprises a salt of phosphate or bicarbonate/carbonate.

In some embodiments, the buffer solution of liquid formulation comprises phosphate family ions, i.e., phosphate (PO₄³⁻), hydrogen phosphate (HPO₄²⁻), and/or dihydrogen phosphate (H₂PO₄⁻).

In some embodiments, the buffer solution of liquid formulation comprises carbonate family ions, i.e, bicarbonate (HCO₃⁻) and carbonate (CO₃²⁻.

In some embodiments, the buffer solution of liquid formulation comprises both phosphate family ions (PO₄³⁻, HPO₄²⁻, and/or H₂PO₄⁻ ions) and carbonate family ions (i.e., HCO₃⁻ and CO₃²⁻.

In some embodiments, the buffer solution of liquid formulation has a buffer salt concentration between about 1 mM and about 100 mM.

In some embodiments, the buffer solution of liquid formulation has a buffer salt concentration between about 5 mM and about 50 mM.

In some embodiments, the buffer solution of liquid formulation has a buffer salt concentration about 10 mM.

In some embodiments, the buffer solution contains sodium or potassium phosphate salts, or a combination thereof.

In some embodiments, the buffer solution contains potassium phosphate; the concentration of the phosphaplatin compound is 5 mg/mL and the pH is in the range of about 7.0 to about 8.0.

In some preferred embodiments, the buffer solution comprises a pyrophosphate salt, for example, sodium pyrophosphate or potassium pyrophosphate.

In some embodiments, the molar ratio of pyrophosphate anion to the phosphaplatin compound is at least 0.1 to 1.

In some embodiments, the molar ratio of pyrophosphate ion to the phosphaplatin compound is about 0.2 to 1 In some embodiments, the molar ratio of pyrophosphate ion to the phosphaplatin compound is about 0.4 to 1.

In a particular preferred embodiment, the concentration of the phosphaplatin compound is about 5 mg/mL, the pyrophosphate concentration is about 5.2 mM, and the pH is in the range of about 7.0 to about 8.0.

As a person of ordinary skill in the art would understand, the present disclosure encompass any reasonable combinations of the embodiments disclosed herein in the same or different aspects.

The term “a,” “an,” or “the,” as used herein, represents both singular and plural forms. In general, when either a singular or a plural form of a noun is used, it denotes both singular and plural forms of the noun.

When the term “about” is applied to a parameter, such as pH, concentration, or the like, it indicates that the parameter can vary by ±10%, preferably within +5%, and more preferably within ±5%. As would be understood by a person skilled in the art, when a parameter is not critical, a number is often given only for illustration purpose, instead of being limiting.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition.

The term “subject” or “patient”, as used herein, refers to a human or a mammalian animal, including but not limited to dogs, cats, horses, cows, monkeys, or the like.

As used herein, any undefined terms take ordinary meaning as would be understood by a person of ordinary skill in the art.

While not intending to be bound by theory, extensive studies have demonstrated that PT-112 mechanism of action involves drug-induced mitochondrial dysfunction, that is, PT-112-induced mitochondrial dysfunction and stress play a significant role in how PT-112 kills cancer cells. These include PT-112-induced mitochondrial ROS and mitochondrial membrane depolarization. Further, while not intending to be bound by theory, PT-112 may disrupt the mevalonate pathway because of the structural similarity of PT-112's pyrophosphate moiety to bisphosphonates. This hypothesis is supported by the observation that PT-112 substantially reduced the amount of ubiquinone (Coenzyme Q10) in the L929 family of cell lines, as several bisphosphonates are known to inhibit the mevalonate pathway, which feeds into the synthesis of ubiquinone.

The following non-limiting examples will illustrate certain aspects of the present invention.

### EXAMPLES

### Example 1

This example describes the materials and methods used in the Examples below.

Cell Culture and Generation of Cybrids Mouse fibroblast cell lines L929 and L929-derived (L929dt) were routinely cultured in high glucose DMEM medium with GlutaMAX (Life Technologies, Paisley, UK) supplemented with 10% of fetal calf serum (FCS), penicillin (1000 U/ml) and streptomycin (10 mg/ml) (PanBiotech, Aidenbach, Germany) at 37° C. and 5% CO₂ using standard procedures. The transmitochondrial cell lines L929^{dt} and dt^{L929} were obtained as previously described (Schmidt, W., et al. (1993) 53:799-805) and cultured with the identical medium used with the parental cells. For L929-ρ⁰ cells, complete DMEM medium was also supplemented with 100 pyruvate (100 μg/ml) and uridine (50 μg/ml).

### Cell Viability Assays

Relative cell growth was measured using the Mossman's method. 3×10⁴ cells were seeded per well in a 96-well flat-bottomed plate and incubated with increasing concentrations of PT-112 or cisplatin (2, 6, and 10 μM) for 24-72 h at 37° C. Then, 10 μl of a 5 mg/ml MTT dye solution was added per well and incubated for 3 hours. During the incubation time, viable cells reduce MTT solution in insoluble purple formazan crystals, solubilized afterwards with isopropanol and 0.05 M HCl mixture and the absorbance was measured in a microplate reader (Dynatec, Pina de Ebro, Spain).

### Cytotoxicity Assays and Cell Death Quantification

Cytotoxicity assays were carried-out as follows: 100 μl aliquots of 3×10⁴ cells were seeded per well in 96-well plate and 10 μM of PT-112 or cisplatin was added and incubated for 24-72 h at 37° C. Cell death was analyzed using a FACScalibur flow cytometer (BD Biosciences) after incubation with Annexin-V-FITC and/or 7-AAD (BD Biosciences, Madrid) in annexin binding buffer (140 mM NaCl, 2.5 mM CaCl₂), 10 mM HEPES/NaOH, pH 7.4) for 10 minutes.

### ROS Production and Mitochondrial Membrane Potential Measurement

Total ROS production and mitochondrial membrane potential were simultaneously measured using a FACScalibur flow cytometer. Pretreated cells with PT-112 were incubated with DiOC₆ at 20 nM (Molecular Probes, Madrid) and DHE at 2 μM (Molecular Probes, Madrid) for 30 min at 37° C. For specific mitochondrial ROS production, cells were incubated with MitoSOX™ (5 μM, ThermoFisher, Rockford, USA) for 30 minutes at 37° C.

### Apoptosis and Necroptosis Inhibition Assays

Cells (3×10⁴) were seeded in a 96-well plate and incubated with a pan-caspase-inhibitor Z-VAD-fmk (50 μM, MedChem Express, New Jersey, USA) and/or RIPK-1 inhibitor necrostatin-1 (30 μM, MedChem Express, New Jersey, USA) for 1 h. After that, cells were treated with 10 μM of PT-112 and incubated for 48 h at 37° C. Both inhibitors were refreshed in their corresponding well after 24 h. Finally, cell death was assessed using flow cytometry after incubation with annexin-V-FITC and 7-AAD for 10 minutes.

### Analysis of Caspase-3 Activation

Caspase-3 activation was measured using an FITC-labelled antibody against cleaved caspase-3 form (BD Pharmingen™, Madrid). For this propose, pretreated cells with 10 μM of PT-112 were fixed with 4% PFA solution for 15 minutes at 4° C. Then, cells were washed with PBS buffer, permeabilized using a 0.1% saponin dilution supplemented with 5% fetal bovine serum and incubated for 15 minutes at room temperature (RT). After washing them, samples were incubated with the antibody for 30 minutes at RT and analyzed by flow cytometry.

Cyto-ID® Analysis. Measurement of Autophagosome Formation

For autophagy analysis, the autophagosome formation after treatment with PT-112 was evaluated using Cyto-ID® probe (Enzo Life Sciences). Pretreated cells with 10 μM of PT-112 were incubated with 1 μl/ml of Cyto-ID® dye reagent for 30 minutes at 37° C. Subsequently, cells were washed with PBS buffer and analyzed by flow cytometry. For autophagy positive controls, cells were treated with 1 μM of rapamycin at least 12 hours before the analysis.

### DAMP Emission

Calreticulin surface expression upon incubation with PT-112 (24-72 h) was analyzed by flow cytometry. PT-112 pretreated cells were incubated with primary rabbit antibody (Abcam, #AB2907, 1:700) at 4° C. for 1 h. Then, cells were washed with PBS and incubated simultaneously with secondary goat antibody anti-rabbit IgG conjugate with Alexa Fluor488® (Invitrogen, #A11034) and 7-AAD. To exclude non-specific interactions, a point of non-treated cells was incubated only with secondary-labelled antibody. 7-AAD positive cells were excluded from the analysis.

ATP secretion was quantified using the luciferase-based kit ENLITEN ATP Assay (Promega). Supernatant of treated cells were collected at different times of incubation (24,48 y 72 h) and ATP concentration was quantified using a fluorometer (Biotek).

### Western-Blot Analysis

Cells (5×10⁶) were lysed with 100 μl of a buffer lysis 1× (1% Triton-X-100; 150 mM NaCl; 50 mM Tris/HCl pH 7,6; 10% v/v glycerol; 1 mM EDTA; 1 mM sodium orthovanadate; 10 mM sodium pyrophosphate; 10 μg/ml leupeptin; 10 mM sodium fluoride; 1 mM methyl phenyl sulfide, Sigma, St. Louis, USA) for 30 minutes in ice. The mixture was centrifugated at 12,000 rpm for 20 minutes at 4° C. The protein concentration in supernatant was analyzed using a BCA assay (Thermo Fisher, Rockford, USA) and was mixed with lysis buffer 3× (SDS 3% v/v; 150 mM Tris/HCl; 0.3 mM sodium molybdate; 30% v/v glycerol; 30 mM sodium pyrophosphate; 30 mM sodium fluoride; 0.06% p/v bromophenol blue; 30% v/v 2-mercaptoethanol, all purchased from Sigma, St. Louis, USA). Protein separation was performed using SDS-PAGE 6 or 12% polyacrylamide gel and then proteins were transferred to nitrocellulose membranes using a semi dry electro transfer (GE Healthcare, Chicago, USA). Membranes were blocked with TBS-T buffer (Tris/HCl 10 mM, pH 8; NaCl 0.12 M; Tween-20 0.1%, thimerosal 0.1 g/L, Sigma, St. Louis, USA) containing 5% skimmed milk. Protein detection was performed by western-blot technique using specific antibodies against p62 (Santa Cruz, SC-28359), LC3BI/II (Sigma, L7543) and HIF-lu (Novus, NB100-479) that were incubated overnight at 4° C. with agitation. Anti-rabbit secondary antibody labeled with peroxidase (Sigma, A9044) was incubated for 1 hour at room temperature with gentle shaking. Proteins were reveled with the reagent Pierce ELC Western Blotting Substrate (Thermo Scientific, Rockford, USA) using Amersham Imager 680 (GE Healthcare Life Sciences).

### Statistical Analysis and Data Processing

Computer-based statistical analysis was performed using GraphPad Prism program (GraphPad Software Inc.). For quantitative variables results are shown as mean±standard deviation (SD). Statistical significance was evaluated using Student t test and differences were considered significant when p≥0.05. Data obtained by flow cytometry were analyzed using FlowJo 10.0.7 (Tree star Inc.).

### Example 2

### Cell Growth Inhibition by PT-112 and Cisplatin in L929, L929dt and Cybrid Cells

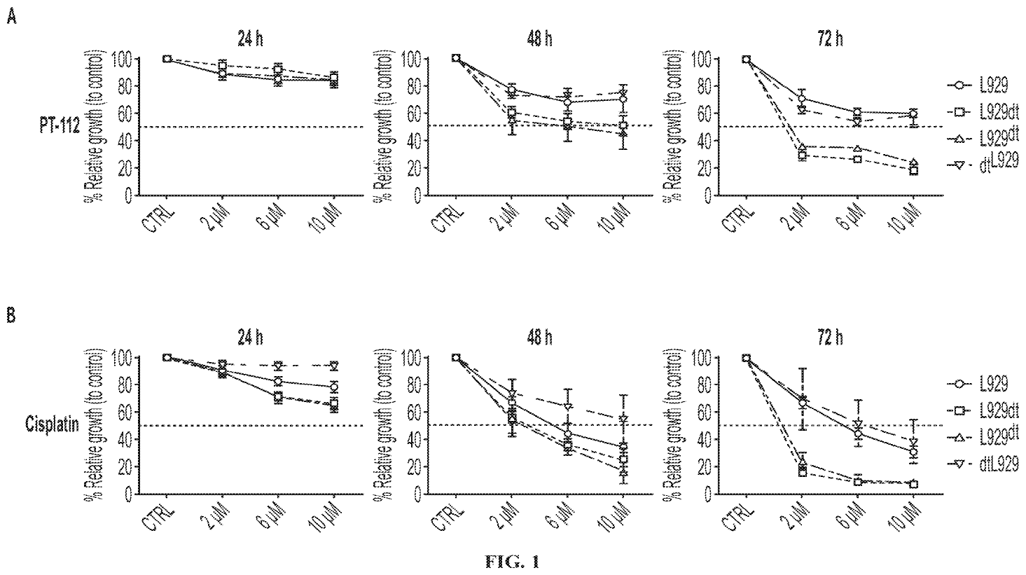

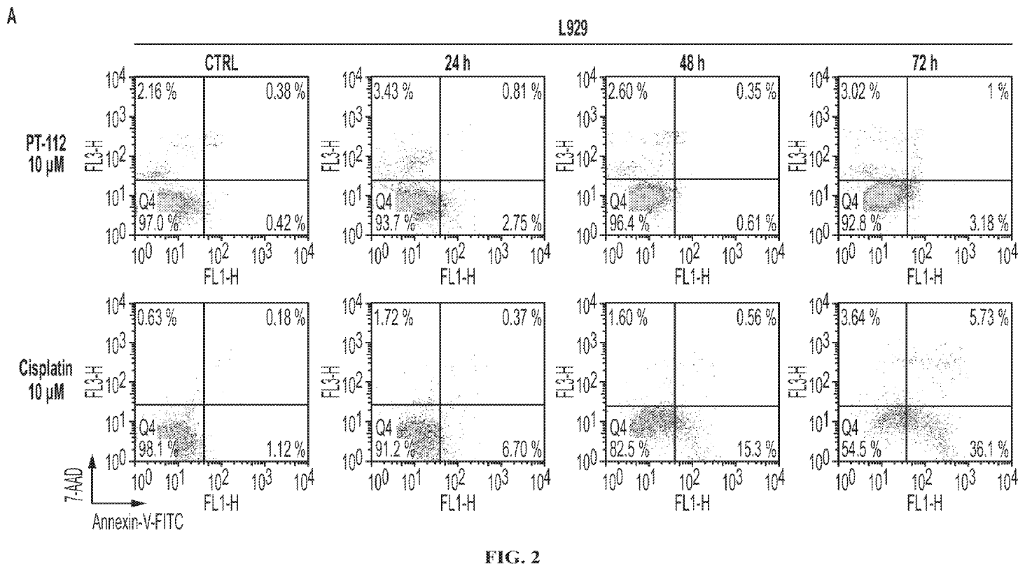

The sensitivity of L929, L929dt and cybrid cells to PT-112 was compared. The parameters studied were compared with those induced by cisplatin, a known Pt-derived chemotherapeutic agent, which mechanism of action involves DNA damage and apoptosis induction (Barry, M., et al. (1990) Biochem Pharmacol, 40, 2353-2362). All cell lines were treated with increasing concentrations of PT-112 or cisplatin (2, 6 and 10 μM) and incubated for 24-72 h at 37° C. (see FIG. 1A and FIG. 1B, which correspond to results obtained with PT-112 and cisplatin incubations, respectively). The doses used are compatible with clinically relevant concentrations, achieved during in vivo treatments (Karp, D., et al. (2018) Ann Oncol, 29, viii143; Bryce, A., et al. (2020) J Clin Oncol, 2020:38). The ability of both drugs to inhibit cell growth was assessed by the MTT reduction method. As shown in FIG. 1A, PT-112 inhibits cell growth in a time-dependent manner, since a clear decrease in cell growth is not observed until 48 hours of exposition. It was observed that the glycolytic cells (L929dt and L929^{dt} cybrid) were more sensitive to PT-112 than L929 cells and the L929^{dt} cybrid. Indeed, this tendency was accentuated at a long-time drug exposure (72 h) in which the growth of Warburg-dependent cells was inhibited by 80% at the highest dose. On the contrary, the slight growth inhibition observed in L929 cells and the L929^{dt} cells (a 40% at the higher dose used) stabilized at 48 h and didn't increase at longer times. In dt^{L929} cybrid cells, the slight growth inhibition observed at 48 h (35% as maximum value) was transient and normal growth was recovered at 72 h.

Regarding cisplatin, a significant effect was clearly observed at short-time exposures that was not observed with PT-112. At 48 h, cisplatin inhibited the growth of all cell lines, with no statistically significant differences between them. At 72 h, the effect at lower concentrations was more pronounced on the more glycolytic cells, but growth at the higher doses was affected in all cell lines (95% inhibition in L929dt and L929^{dt} cells and 70% inhibition in L929 and dt^{L929} cells). These data demonstrate that PT-112 has a marked selectivity on especially glycolytic tumor cells, confirming our hypothesis on a mechanism of action related with the metabolic status of tumor cells, while cisplatin is less selective and acts through a different mechanism.

### Example 3

### Cytotoxic Effect of PT-112 and Cisplatin in L929, L929dt and Cybrid Cells

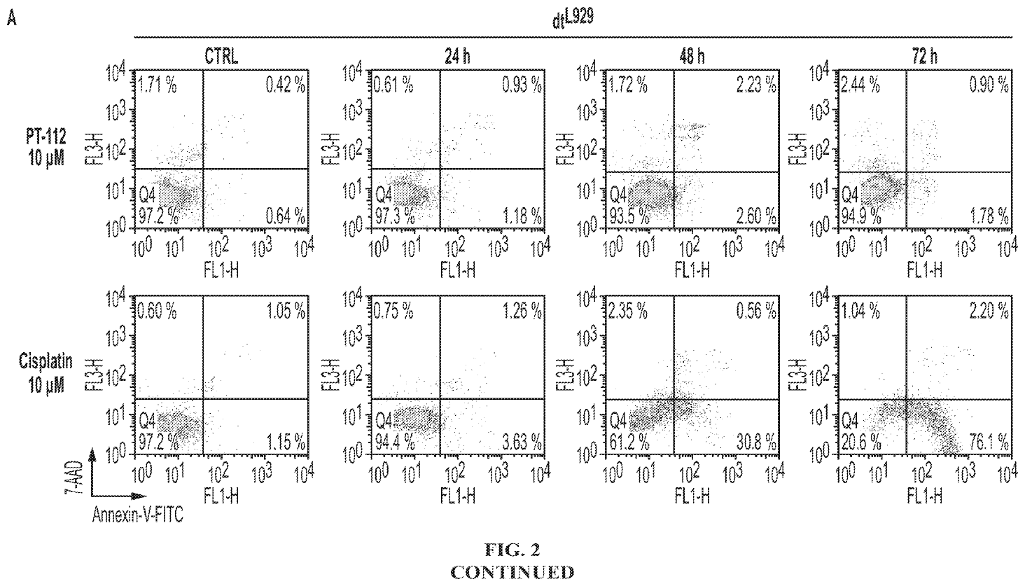

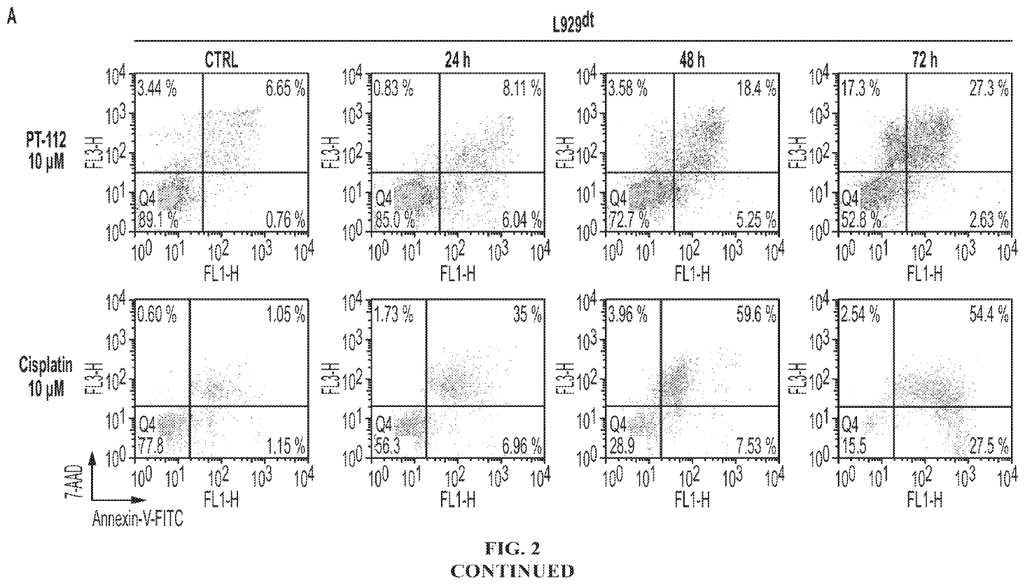

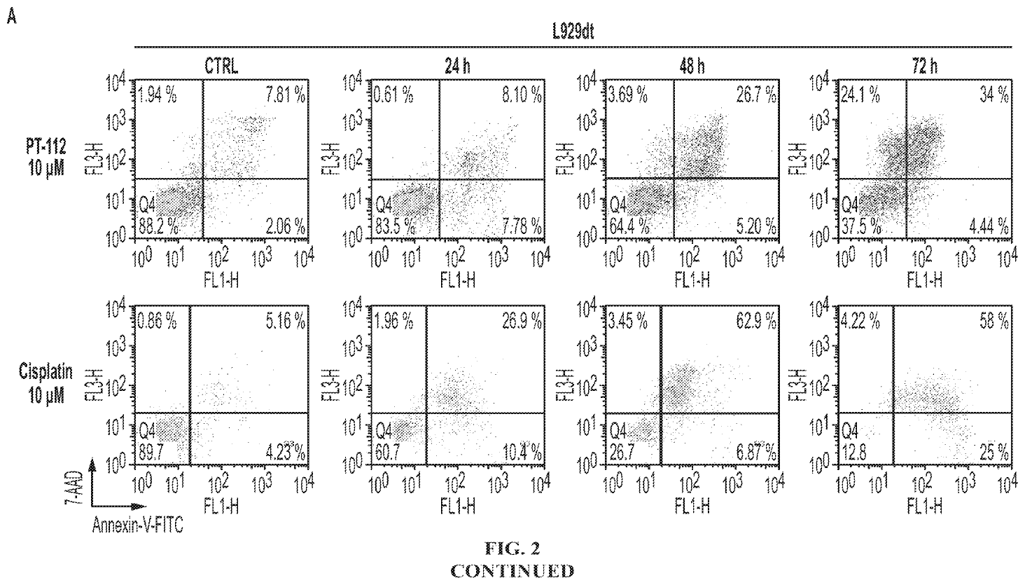

To test cell death induction by PT-112 and cisplatin, the parental cells L929, L929dt and cybrids cells were incubated with 10 μM of PT-112 or cisplatin for 24, 48 or 72 h and, at the end of the incubations, simultaneously stained with annexin-V-FITC and 7-AAD and analyzed by flow cytometry. See FIG. 2A, where dot-plots represent the staining evolution of treated cell population compared to the control, and FIG. 2B shows graph-bars, which correspond to a graphical representation of obtained data remarking cell percentage in each quadrant of dot-plot figures. The results are shown as mean±SD of at least 2 independent experiments made in duplicate. The results obtained indicate that cisplatin induces cell death in all cell lines, especially after long-time drug exposure and exerts cytotoxicity faster than PT-112 (FIG. 2A). On the contrary, PT-112 was cytotoxic only on highly glycolytic cells, indicating a high selectivity of action and correlating with data shown in FIG. 1 (FIG. 2A). Regarding the annexin-V-FITC and 7-AAD staining pattern, in cisplatin-induced cell death, a population of annexin-V⁺ but 7-AAD⁻ cells, characteristic of apoptotic cell death, was observed in all cell lines, albeit cell death was executed more rapidly in the most glycolytic cells (FIG. 2B, bar sections colored in black). On the contrary, in cells treated with PT-112, this population is not observed at any time point in sensitive L929dt and L929^{dt} cells, and a population double positive for both markers is at short times of exposure, increasing with time (FIG. 2B, bar sections colored in white). Finally, at longer times, a population positive for 7-AAD and negative for annexin-V staining appears for both cell lines, typical of necrotic cell death (FIG. 2B, sections colored in grey). Taken together, these results clearly demonstrate that the mechanism of action and the selectivity of cisplatin and PT-112 are completely different. While cisplatin seems to follow the canonical apoptotic pathway used by many chemotherapeutic drugs, such as doxorubicin (Gamen, S., et al. (1997) FEBS Lett., 417:360-364; Gamen, S., et al. (2000) Exp. Cell Res., 258:223-235), PT-112 does not comply with this canonical pathway, showing some hints of necrotic cell death.

### Example 4

PT-112 Disturbs Mitochondrial Membrane Potential and Induces Caspase-3 Activation, but Caspase Inhibition Did not Protect from Cell Death

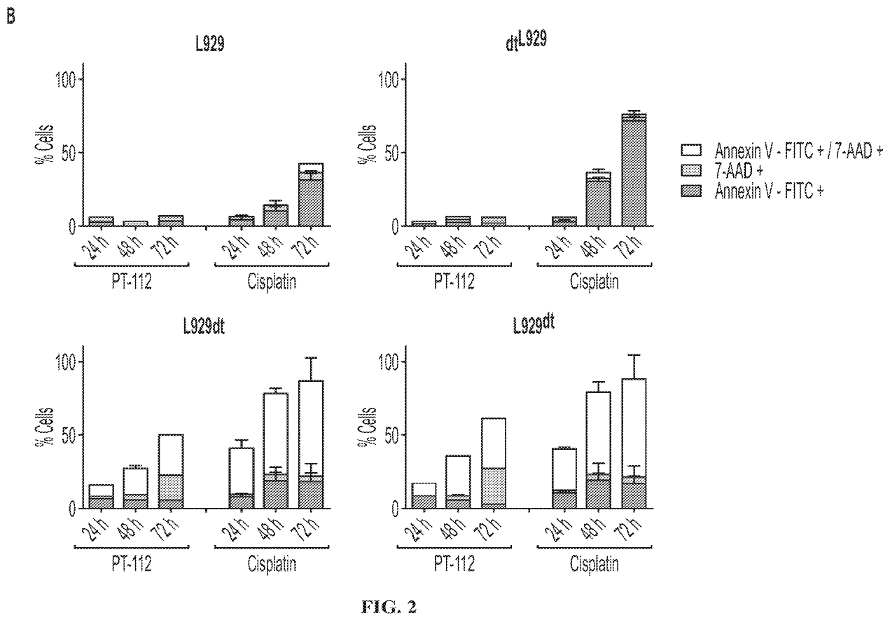

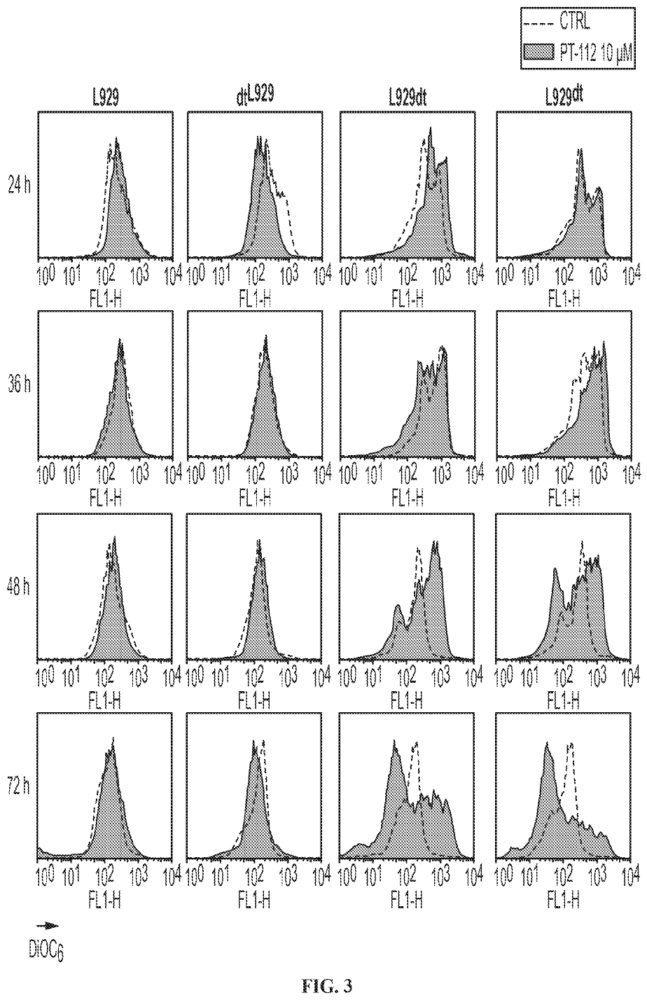

Another typical event related with the activation of the mitochondrial apoptotic pathway is the loss of mitochondrial membrane potential (ΔΨₘ); thus, the effect of PT-112 on ΔΨₘ was analyzed using DiOC₆ staining and flow cytometry. As shown in FIG. 3, while ΔΨₘ did not suffer any change during the 72 h incubation with PT-112 in L929 and dt^{L929} cells, a very significant and characteristic effect was observed in sensitive glycolytic cells. Remarkably, ΔΨₘ increased in these cells upon PT-112 treatment, instead of directly decreasing, as should it happen in a typical apoptosis process. The appearance of a population of cells with hyperpolarized mitochondria at 48 h was observed, simultaneously accompanied by a population that partially lost ΔΨₘ. At 72 h, both populations can be still detected, but that with low ΔΨₘ became predominant.

### Example 5

PT-112 Induces Caspase-3 Activation but Z-VAD-Fmk and Necrostatin-1 Did not Protect from Cell Death

Although data indicate that PT-112 does not kill sensitive cells through a typical apoptotic process, PT-112's effect on caspase-3 activation, the main apoptotic executor, was analyzed. For this purpose, a FITC-labelled anti-caspase-3 antibody that detects cleaved, active caspase-3 by flow cytometry was used. Cells (3×10⁴) were treated with 10 μM of PT-112 for 24-72 h. Then, cells were incubated with anti-cleaved caspase-3 labelled with FITC dye and analyzed by flow cytometry. As shown in FIG. 4A the levels of active caspase-3 clearly increased in a time-dependent manner in glycolytic cells sensitive to PT-112-induced cell death. The implications of caspase-3 activation in this process were investigated by tested the ability of the general pan-caspase inhibitor Z-VAD-fmk and/or the necroptosis inhibitor necrostatin-1 to prevent cell death induced by PT-112 (FIG. 4B). Cells were pretreated for 1 h with or without pan-caspase or/and necroptosis inhibitors and then, incubated with 10 μM of PT-112 for 48 h. Flow cytometry analysis was carried-out using annexin-V-FITC and 7-AAD staining. Cells were stained simultaneously with annexin-V-FITC and 7-AAD and the percentage of the different populations analyzed by flow cytometry. In agreement with results presented in FIG. 2A, PT-112 induces a direct accumulation of double positive cells. Z-VAD-fmk, necrostatin-1 or their combination did not inhibit cell death, and the double positive population remained the largest subset in all cases. In fact, cells treated with PT-112 in the presence of Z-VAD-fmk increased their mortality rate compared to PT-112 alone; notwithstanding, necrostatin-1 did prevent this increase, without affecting the rate of cell death induced by PT-112. This observation indicates the presence of a necroptotic component, but only if caspases are inhibited, reminiscent of other cell death inducers such as TNF-α in L929 cells (Vercammen, D., et al., (1998) J. Exp. Med., 187:1477-1485).

### Example 6

PT-112 Induces Massive Mitochondrial Reactive Oxygen Species (ROS) Production in Sensitive Cells

In order to obtain more evidence about the mechanism of action of PT-112, its effect on ROS production was analyzed. First, a time-course determination of total ROS generation by detection of 2HE oxidation was performed by flow cytometry. As shown in FIGS. 5A-5B, a moderate increase was observed in total ROS production in a time-dependent manner in all cell lines tested, reaching maximum levels between 48 and 72 h. L929 and dt^{L929} cells showed similar levels of total ROS production than L929dt and L929^{dt} cells after 72 h or exposure, but the increase in ROS levels was detected faster in the glycolytic cells. To complete this study, specific mitochondrial ROS production upon PT-112 treatment using the MitoSOX™ reagent was determined. As shown in FIG. 5C, mitochondrial ROS production was massively increased only in sensitive cells after treatment with PT-112 and only barely in L929 or dt^{L929} cells, suggesting that this event is specifically involved in cell death induced by PT-112.

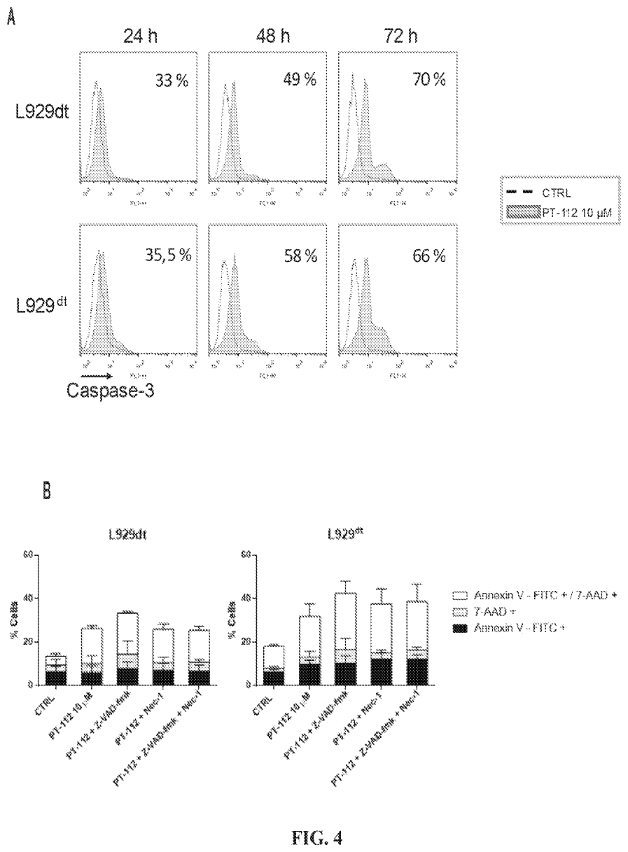

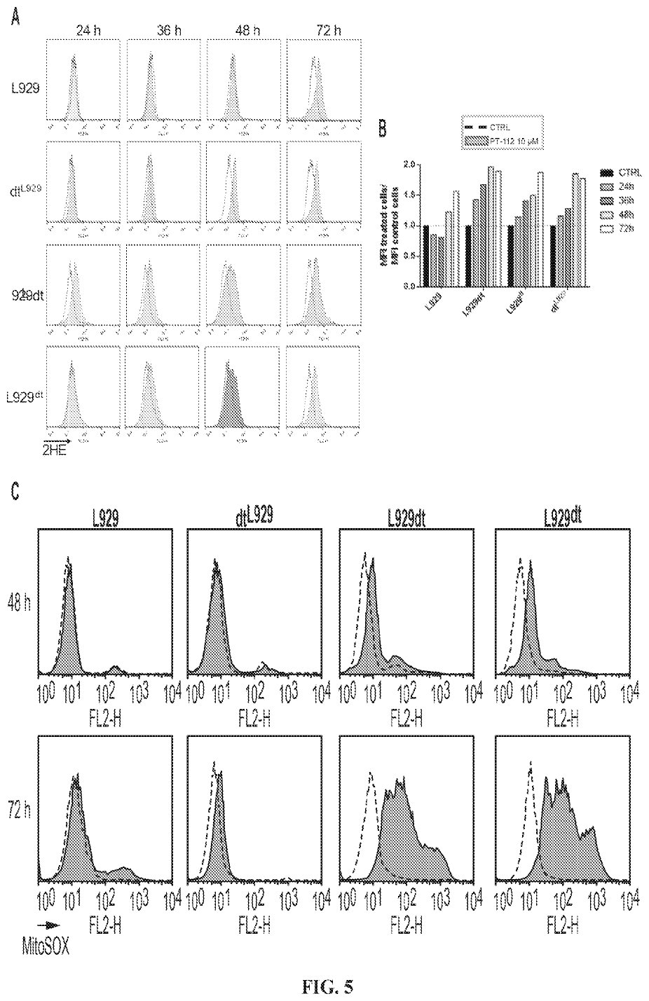

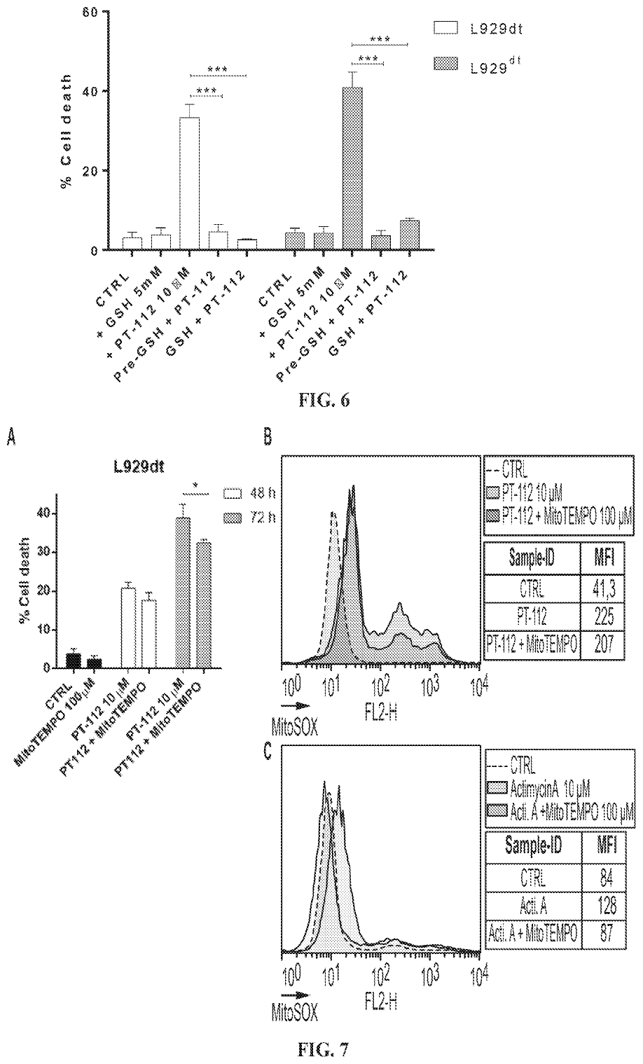

Next, the implication of ROS generation in the cell death process induced by PT-112 was demonstrated using a variety of ROS scavengers. Treatment with glutathione (GSH) completely abolished PT-112-induced cell death in L929dt and L929^{dt} cells (FIG. 6). However, this effect can be due to direct reactivity of the thiol group with Pt, inactivating the cytotoxic potential of PT-112, and not to the elimination of ROS generation. This hypothesis was confirmed by incubating PT-112 with GSH during 1 h in the absence of cells and adding this GSH-treated PT-112 to cells, showing no cytotoxicity (FIG. 6). Hence, other ROS scavengers that did not contain a thiol group were studied, such as the chemical superoxide dismutase mimetic MnTBAP, the piperidin TEMPO, or the ROS scavenger in the lipid phase α-tocopherol (vitamin E), but neither of them were able to prevent PT-112-induced cell death in sensitive cells (data not shown). Since PT-112-induced ROS generation seems to be concentrated in mitochondria, the mitochondria-specific ROS scavenger MitoTEMPO was used. Cells (3×10⁴) were seeded in a 96-well plate in phenol red-free medium and were incubated with 100 μM of MitoTEMPO for 2 h. Then, 10 μM of PT-112 was added and incubated for 48 and 72 h at 37° C. As shown in in FIG. 7C, MitoTEMPO was able to almost completely abolish mitochondrial superoxide generation induced by the mitochondrial complex III inhibitor, antimycin A. Regarding PT-112, the amount of mitochondrial superoxide anion was much higher than that induced by antimycin A (compare the MFI values, from 128 to 225), and MitoTEMPO was able to inhibit this event, albeit only partially (FIG. 7B). This same partial protection was also observed for PT-112-induced L929dt cell death, being statistically significant after 72 h (FIG. 7A). These data indicate that mitochondrial ROS generation is implicated in PT-112-induced cell death but being difficult to prevent by chemical means.

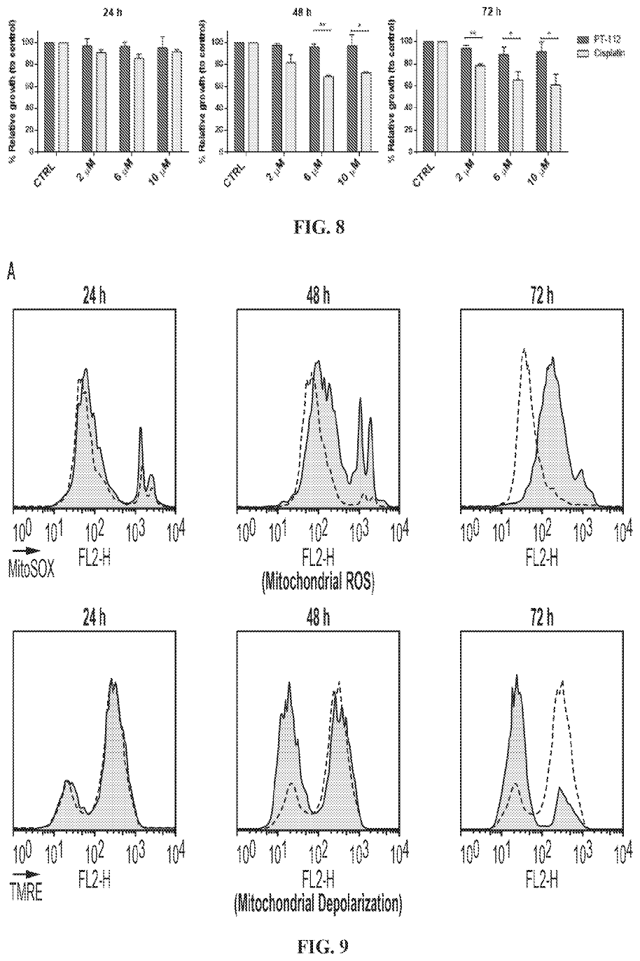

As an alternative approach, L929-ρ⁰ cells were used. ρ⁰ Cells are devoid of mtDNA by prolonged exposure to ethidium bromide and are unable to perform OXPHOS or to generate mitochondrial ROS, although upon specific treatments, such as perforin/granzyme B, are able to generate ROS from extramitochondrial sources (Aguiló, J. I., et al.; Cell Death Dis 2014, 5, e1343; Catalán, E., et al.; OncoImmunol 2015, 4, e985924). The growth inhibition effect of PT-112 and cisplatin on L929-ρ⁰ cells were tested, as done in FIG. 1 for the L929-derived cell lines used in this study. As shown in FIG. 8, while cisplatin inhibited the growth of these cells, PT-112 scarcely affect their growth rate at any concentration or time of incubation. PT-112 was also almost unable to induce cell death on L929 or dtL929 cells (FIG. 2), but it did inhibit the growth of these cells (FIG. 1), while it was without effect on L929-ρ⁰ cells. These data, together with the partial inhibition of cell death achieved by MitoTEMPO, point to the observed massive mitochondrial ROS generation as a central event in PT-112-induced cell death.

This phenomenon was observed in a panel of mouse cell lines, namely RWPE-1, 22RV1, PC-3, LNCap-C4-2, LNCap, DU-145, and LNCap-C4 with an increasing PT-112 sensitivity, where those with mitochondrial mutations were more suspectable to PT-112 and had large increases in mtROS. The different effects of cisplatin seen in this cell line versus PT-112 provide more evidence of the substantial differences between these two drugs. Additionally, in a large panel of prostate cancer cell lines the same pattern of overlapping PT-112 sensitivity and mtROS generation was seen, corroborating these phenomena in relevant human cancer cells.

### Example 7

PT-112 Induces Massive Mitochondrial Membrane Depolarization

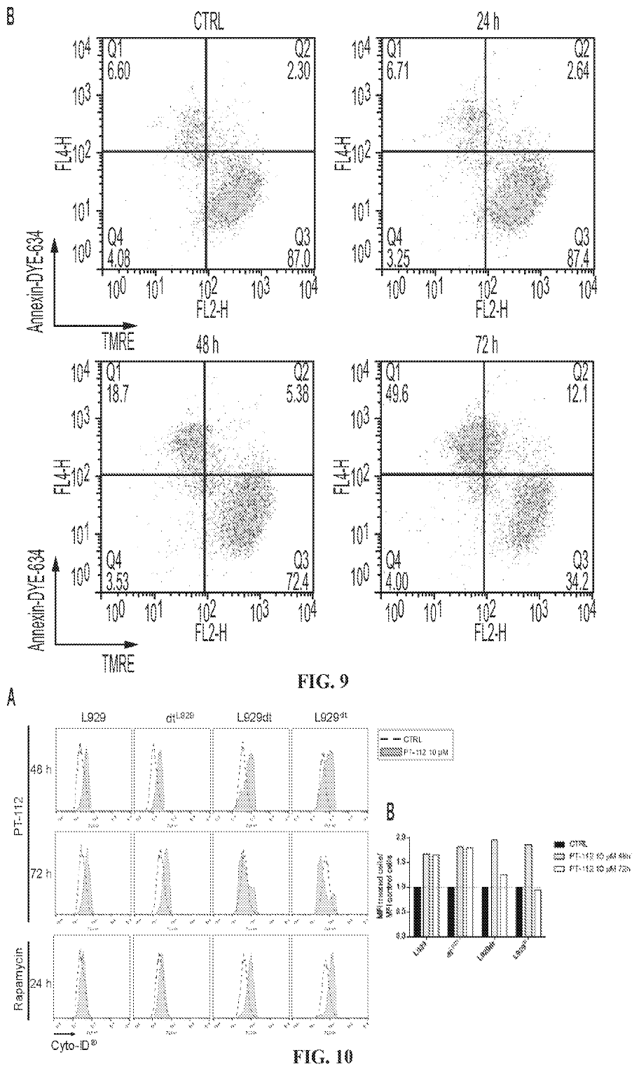

Another sign of mitochondrial stress and dysfunction is mitochondrial membrane depolarization, which can be captured via flow cytometry. It was observed that PT-112 induces this concurrently with the mtROS accumulation, and again in cell lines that are PT-112 sensitive, specifically LNCap-C4 Prostate Cancer Cell Line (FIG. 9A). This second line of evidence further solidifies our understanding that mitochondrial dysfunction is an important aspect of PT-112's mechanism.

Flow cytometry has established that loss in mitochondrial membrane potential indeed correlates over time with cell death and allows us to visualize such loss in mitochondrial activity across the same time points (FIG. 9B). With this work, PT-112's effects on mitochondria in sensitive cells appear to be important to its cytotoxic effects.

### Example 8

PT-112 Induces Autophagosome Formation

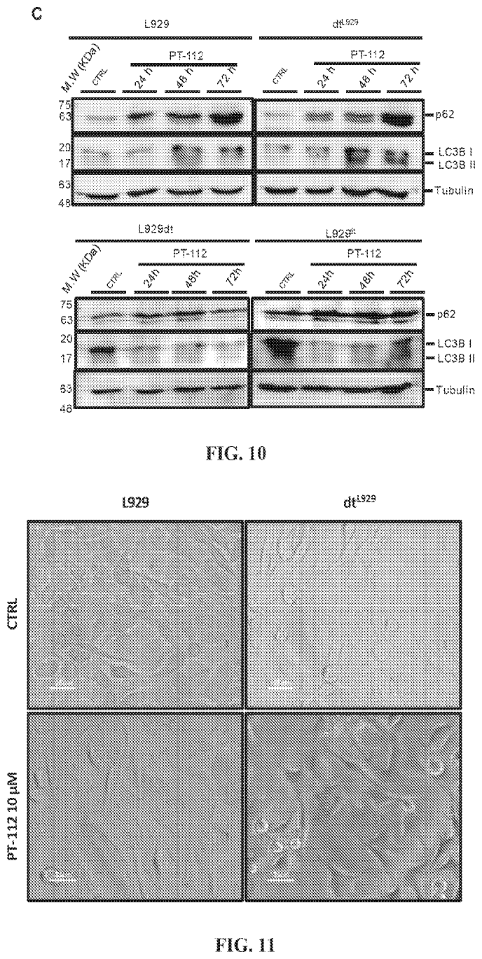

After assessing that PT-112 did not induce canonical apoptosis or necroptosis, the possibility that it could induce autophagy was tested. The initiation of autophagy was analyzed using the Cyto-ID® method that allows detection of intracellular autophagosome formation by flow cytometry. As shown in FIG. 10A and FIG. 10B, PT-112 clearly induces autophagosome formation in all cell lines at 48 h of PT-112 treatment. At 72 h, autophagosome formation apparently decreased in L929dt and L929^{dt} cells, possibly due to the induction of cell death. On the contrary, in L929 and dt^{L929} cells, autophagosome formation is maintained at 72 h, likely explained by the lack of cell death was observed in these lines. Of note, it was observed that glycolytic cells were more sensitive to autophagy induction by rapamycin than L929 and dt^{L929} cells (FIG. 10). To further investigate the activation of autophagy upon PT-112 treatment, expression levels of p62 and the conversion of LC3BI to LC3BII, known indicators of autophagy induction were analyzed. The results obtained (FIG. 10C) show a clear conversion of LC3BI to LC3BII in L929 and dt^{L929} cells, and a gradual accumulation of p62. In glycolytic cells, no significant changes were observed in p62 levels, but a rapid reduction in LC3BI levels was observed, which was accompanied by the appearance of faint LC3BII bands. The Cyto-ID® results, the most sensitive method to detect autophagosome formation, and the LC3B data demonstrate that PT-112 induces the initiation of the autophagy process. However, the absence of p62 reduction or degradation indicates that the autophagic process does not conclude.

### Example 9

Cell Morphology after PT-112 Treatment

The observation of cells on the microscope after treatment with PT-112 showed abundant brilliant spots inside cytoplasm of the four cell lines, that could well correspond to autophagosomes. In addition, in L929dt and L929^{dt} cells, sensitive to cell death induction by PT-112, an enormous amount of small, uniform cell debris was also detected (FIG. 11). This spreading of tumor corpses corresponds to danger signals emission by dead tumor cells, in agreement with the described immunogenic nature of PT-112-induced cell death (Yamazaki, T., et al. (2020) Oncolmmunol, 9, e1721810.).

### Example 10

### Effect of PT-112 on Mitochondrial CoQ10 Levels

In order to test the possible effect of PT-112 on enzymes of the mevalonate pathway, the prenylation state of the chaperone HDJ-2 or of the small GTPases implicated in vesicular traffic Rab5 and Rab7 was tested. However, no clear effects on prenylation were observed using this approach (data not shown). The mevalonate pathway not only provides farnesyl or geranylgeranyl units for protein post-translational modifications, but also provides longer prenyl groups for the final steps of Coenzyme Q synthesis, generating coenzyme Q9, Q10 or longer ubiquinone derivatives (Gruenbacher, G. et al.; OncoImmunol 2017, 6, e1342917; Tricarico, P., et al.; Int JMol Sci 2015, 16, 16067-16084). In all these steps of the mevalonate pathway pyrophosphate derivatives are central for enzyme activity, and PT-112 could act on these enzymes through its pyrophosphate moiety.

### Example 11

### Effects of PT-112 on Rab5 Prenylation and Dimer Formation

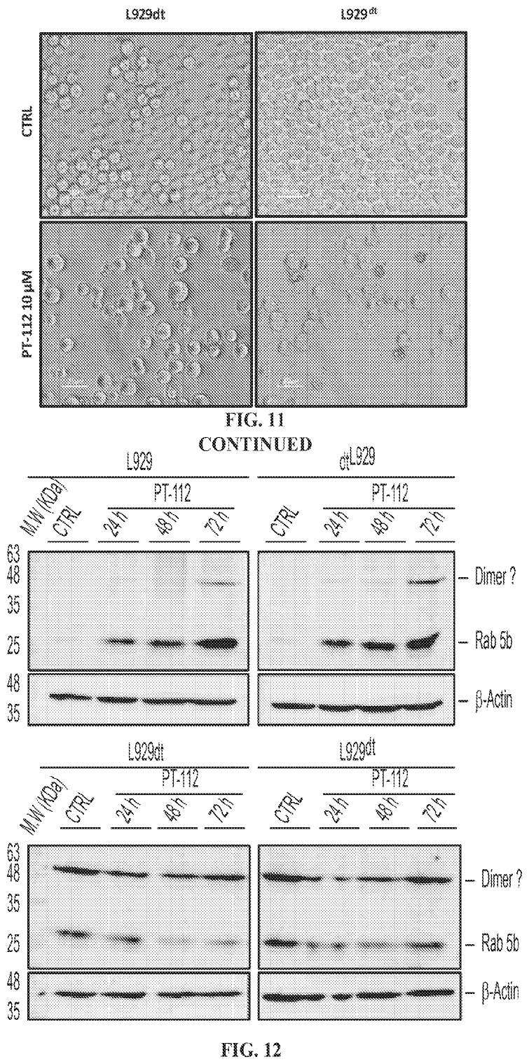

In order to test the possible effect of PT-112 on enzymes of the mevalonate pathway, the prenylation state of the small GTPase implicated in vesicular traffic Rab5 was tested. As shown in FIG. 12, the treatment of L929 and dt^{L929} cells with PT-112 induced a dramatic increase in the expression of this protein, already observed at 24 h, with a higher mobility band appearing at longer incubation times. This higher mobility band corresponds to the unprenylated protein. In addition, the appearance of this band correlated with the detection of a band with a molecular weight corresponding to the double of Rab5, which could correspond to a Rab5 dimer.

In sensitive glycolytic cells, this possible Rab5 dimerization product was expressed at a high level already at the basal level. The appearance of the higher mobility band was observed especially after 24 h of exposure to PT-112, while at longer times, a net reduction in the expression of Rab5 was observed. However, the band corresponding to the Rab5 dimer did not change upon PT-112 treatment.

### Example 12

### Cells Sensitive to PT-112 Express High Levels of HIF-1α

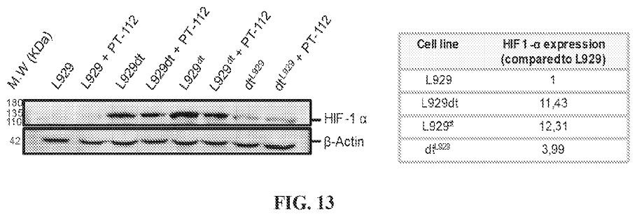

To further investigate the relationship between the glycolytic profile of the L929dt and L929^{dt} cells and hypoxic markers, the expression levels of HIF-lu in our cellular models at basal level, and also after treatment with PT-112 were analyzed. FIG. 13 has demonstrated that even in presence of oxygen, the L929dt and L929^{dt} cells express HIF-lu four-fold greater than the parental L929 and dt^{L929} cells (around a 12-fold increase compared with parental L929 cells). PT-112 did not substantially affect to the low levels of HIF-lu in L929 or dtL929 cells or to the high levels in L929dt and L929dt cells. These data show that sensitivity to PT-112 is closely related with HIF-1α expression, an observation that should have prognostic and clinical applications.

### Discussion

Over the last several decades, new platinum drugs have been developed in order to increase their antitumor potential, avoid resistances and reduce toxicities. These new improved platinum drugs include oxaliplatin (1R,2R-diaminocyclohexane oxalato-platinum (II), based on the 1,2-diaminocyclohexane (DACH) carrier ligand that was originally described in the late 1970s (Kidani, Y., et al. (1978) J Med Chem, 21:1315-1318) and was proposed as a strategy to link a platinum-based drug to a biocompatible water-soluble co-polymer (Kelland, L., (2007) Nature Rev Cancer, 7:573-584.). Consequently, DACH ligand has been employed to design new platinum analogs with the aim of improving their antitumor activity and increase the efficiency of Pt²⁺ delivering to DNA (Schmidt, W., et al. (1993) Cancer Res, 53, 799-805; Rice, J., et al (2006) Clin Cancer Res, 12:2248-2254). Indeed, PT-112 formula is based on the DACH strategy, but it is unique because it contains a pyrophosphate moiety. This unique characteristic gives it a marked bone tropism, that oxaliplatin does not exhibit (Bose, R. et al. (2008) Proc. Natl. Acad. Sci. USA, 105:18314-18319). Regarding its mechanism of action, it has been shown that DNA is not a major target for PT-112 (Bose, R., et al. (2008) Proc. Natl. Acad. Sci. USA, 105:18314-18319; Corte-Rodriguez, M., et al. (2015) Biochem Pharmacol, 98:69-77).

One of the anabolic pathways that are extremely active in tumor cells is the pentose phosphate pathway, needed for the synthesis of DNA and RNA nucleotides (Patra, K., et al. (2014) Trends Biochem. Sci., 39:347-354) and also the mevalonate pathway, needed for the de novo synthesis of sterols and geranyls (Bathaie, S., et al. (2017) Curr Mol Pharmacol, 10:77-85). Famesyl and geranylgeranyl backbones are needed for the post-translational modification of proteins relevant in signaling such as Ras (Tricarico, P., et al. Crovella, S.; Celsi, F., (2015) Int J Mol Sci 2015, 16, 16067-16084.) and also for the synthesis of mitochondrial coenzyme Q derivatives (Tricarico, P., et al. (2015) Int JMol Sci, 16:16067-16084). Both pathways have some steps in which pyrophosphate is needed for correct enzyme activity. In any case, this subject has not been studied in depth in the field of cancer treatment, probably because there were few drugs with a pyrophosphate component. Our hypothesis was that the activity and selectivity of PT-112, due to its pyrophosphate moiety, could have to do with its increased uptake by tumor cells that are especially glycolytic and dependent on the mevalonate pathway, something that will also explain its activity on prostate tumors, multiple myeloma and on bone metastasis.

This hypothesis has been clearly confirmed in this murine model. Glycolytic tumor cells presenting mutations in mtDNA (L929dt and L929^{dt} cybrid cells) are especially sensitive to cell death induced by PT-112 while tumor cells with an intact Oxphos pathway (L929 and dt^{L929} cybrid cells) are less sensitive to PT-112. As a control, all cells are sensitive to the classical Pt-containing drug cisplatin. While cisplatin seems to follow the canonical apoptotic pathway used by many chemotherapeutic drugs, such as doxorubicin (Gamen, S., et al. (1997) FEBS Lett., 417:360-364; Gamen, S., et al. (2000) Exp. Cell Res., 258:223-235.), PT-112 does not comply with this canonical pathway, showing some hints of necrotic cell death.

Whereas PT-112 does not affect mitochondrial membrane potential (ΔΨₘ) in non-sensitive cells, this parameter is changed in sensitive cells in an unconventional way. After short incubation times with PT-112 (24-36 h), an initial mitochondrial hyperpolarization is observed. At longer times (48 h), two cell populations are detected: one with hyperpolarized mitochondria and another one that show loss of ΔΨₘ. At 72 h, this last population predominates, at the same time that cell death is maximal. PT-112 induces caspase-3 activation at the same time as cell death but the general caspase inhibitor Z-VAD-fmk does not inhibit PT-112-induced cell death, alone or in combination with the necroptosis inhibitor necrostatin-1. PT-112 induces reactive oxygen species (ROS) in all cells tested, regardless of their sensitivity to cell death induction, although ROS appears more rapidly in more sensitive cells. However, when this analysis was restricted to the detection of mitochondrial ROS, only the most damaged cells showed a massive mtROS accumulation. This disclosure has demonstrated a partial protection from PT-112-induced cell death in sensitive cells by the use of the mitochondria-restricted ROS scavenger MitoTEMPO. In addition, L929-ρ⁰ cells, devoid of mtDNA and unable to perform OXPHOS or to generate mitochondrial ROS (Catalin, E., et al.; OncoImmunol 2015, 4, e985924.), are completely insensitive to PT-112-induced cell death or growth inhibition. These data point to the observed massive mitochondrial ROS generation as a central event in PT-112-induced cell death.

On the other hand, PT-112 induces the initiation of autophagy in all cell lines, detected by the Cyto-ID® method and by reduction in LC3B I levels. Despite this, it seems that the autophagy process is not completed, since p62 is not degraded.

Taking into account precedents of bisphosphonates activity (Farrell, K., et al. (2017) Bone Rep, 9, 47-60; Qiu, L., et al. (2017) Eur J Pharmacol, 810:120-127), one of the favored hypothesis would be that PT-112 could act directly on enzymes of the mevalonate pathway, such as farnesyltransferase or geranylgeranyl transferase. In fact, it has been reported increases in farnesyltransferase expression activity in prostate cancer patients, correlating with bad prognosis (Todenhofer, T., et al. (2013) World J Urol, 31:345-350), and PT-112 has shown extremely good activity in late stage castration resistant prostate cancer (CRPC), either alone (Karp, D., et al. (2018) Ann Oncol, 29, viii143) or in combination with avelumab (Bryce, A., et al. (2020) J Clin Oncol, 2020:38). Even more, Qiu et co-workers (Qiu, L., et al. (2017) Eur J Pharmacol, 810:120-127) have developed [Pt(en)]₂ZL, a complex which conjugates the bisphosphonate zoledronic acid with Pt²⁺ ions and demonstrated that it prevented the prenylation of small G proteins through inhibition of the mevalonate pathway.

PT-112 activity on farnesyl or geranylgeranyl transferases has not been clearly demonstrated, indicating that its mechanism of action could be different to that described for bisphosphonates. The mevalonate pathway not only provides farnesyl or geranylgeranyl units for protein post-translational modifications, but also provides longer prenyl groups for the final steps of Coenzyme Q synthesis, generating coenzyme Q9, Q10 or longer ubiquinone derivatives (Gruenbacher, G., et al.; OncoImmunol 2017, 6, e1342917; Tricarico, P., et al.; Int J Mol Sci 2015, 16, 16067-16084). In all these steps of the mevalonate pathway, pyrophosphate derivatives are central for enzyme activity, and PT-112 could act on these enzymes through its pyrophosphate moiety.

Finally, the expression of HIF-lu is much higher in glycolytic cells especially sensitive to PT-112 than in cells with an intact OXPHOS pathway. In fact, low levels of CoQ10, as those detected in L929dt cells at the basal state, have been recently correlated with high HIF-lu expression and stabilization (Liparulo, I., et al.; FEBS J 2021, 288, 1956-1974). As a consequence of these observations, HIF-lu expression should be a marker of sensitivity to PT-112 with future clinical applications, as overcoming hypoxia-related tumor resistance and poor outcomes is considered a major objective of contemporary drug development in cancer.

It is understood that the examples and embodiments described herein are for illustrative purposes only and that various modifications or changes in light thereof will be suggested to persons skilled in the art and are to be included within the spirit and purview of this application and scope of the appended claims. All patent or non-patent references mentioned herein are incorporated by reference in their entireties.

1. A method of diagnosing a cancer patient for treatment with a phosphaplatin compound, comprising measuring expression of HIF-1α in glycolytic cells of the cancer patient, wherein an expression of HIF-1α at or above a defined level indicates that the cancer patient can be treated with the phosphaplatin compound effectively.

2. A method of treating a cancer in a patient, comprising the steps of:
(a) measuring expression level of HIF-1α in glycolytic cells of the patient; and
(b) if the expression of HIF-1α in the glycolytic cells obtained in the step (a) is at or above a defined level, administering to the patient a therapeutically effective amount of a phosphaplatin compound.

3. The method of claim 1, wherein the defined level is 1.2 times, 1.5 times, 2.0 times, 2.5 times, 3.0 times, 3.5 times, 4.0 times, 5.0 times, or 6.0 times expression level of HIF-1α in parental cells.

4. A method of inhibiting proliferation of tumor cells characterized by a highly glycolytic phenotype, comprising contacting the cells with a phosphaplatin compound.

5. The method of claim 4, wherein the highly glycolytic phenotype is characterized by an expression level of HIF-1α in glycolytic cells is at least 1.2 times, at least 1.5 times, at least 2.0 times, at least 2.5 times, at least 3.0 times, at least 4.0 times, at least 5.0 times, or at least 6.0 times of expression level of HIF-1α in parental cells.

6. The method of claim 3, wherein the phosphaplatin compound has a structure of formula I or II. 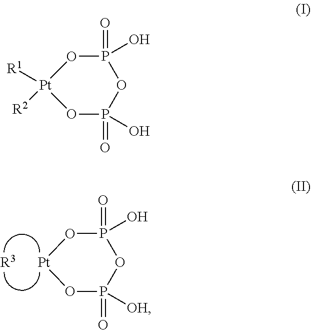
or a pharmaceutically acceptable salt thereof, wherein R¹ and R² are each independently selected from NH₃, substituted or unsubstituted aliphatic amines, and substituted or unsubstituted aromatic amines; and wherein R³ is selected from substituted or unsubstituted aliphatic diamines, and substituted or unsubstituted aromatic diamines.

7. The method of claim 6, wherein R¹ and R² are each independently selected from NH3, methyl amine, ethyl amine, propyl amine, isopropyl amine, butyl amine, cyclohexane amine, aniline, pyridine, and substituted pyridine; and R³ is selected from 1,2-ethylenediamine and cyclohexane-1,2-diamine.

8. The method of claim 6, wherein the phosphaplatin compound is selected from the group consisting of: 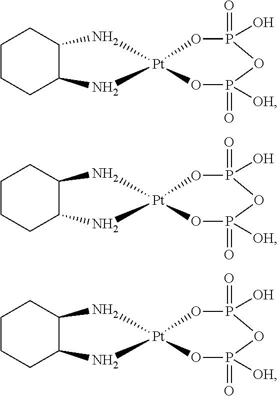
or pharmaceutically acceptable salts, and mixtures thereof.

9. The method of claim 6, wherein the phosphaplatin compound is (R,R)-1,2-cyclohexanediamine-(pyrophosphato)platinum(II) (or “PT-112”), or a pharmaceutically acceptable salt thereof.

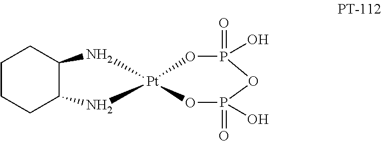

10. The method of claim 3, wherein the cancer or tumor is selected from the group consisting of gynecological cancers, genitourinary cancers, lung cancers, head-and-neck cancers, skin cancers, gastrointestinal cancers, breast cancers, bone and chondroital cancers, soft tissue sarcomas, thymic epithelial tumors, and hematological cancers.

11. The method of claim 10, wherein the bone or blood cancer is selected from the group consisting of osteosarcoma, chondrosarcoma, Ewing tumor, malignant fibrous histiocytoma (MFH), fibrosarcoma, giant cell tumor, chordoma, spindle cell sarcomas, multiple myeloma, non-Hodgkin lymphoma, Hodgkin lymphoma, leukemia, childhood acute myelogenous leukemia (AML), chronic myelomonocytic leukaemia (CMML), hairy cell leukaemia, juvenile myelomonocytic leukaemia (JMNL), myelodysplastic syndromes, myelofibrosis, myeloproliferative neoplasms, polycythaemia vera, and thrombocythaemia.

12. The method of claim 11, wherein the bone or blood cancer is selected from the group consisting of osteosarcoma, chondrosarcoma, Ewing tumor, malignant fibrous histiocytoma (MFH), fibrosarcoma, giant cell tumor, chordoma, spindle cell sarcomas, multiple myeloma, non-Hodgkin lymphoma, Hodgkin lymphoma, leukemia.

13. The method of claim 3, in conjunction with administering to the subject a second anti-cancer agent.

14. The method of claim 13, wherein the second anti-cancer agent is selected from the group consisting of alkylating agents, glucocorticoids, immunomodulatory drugs (IMiDs), proteasome inhibitors, and checkpoint inhibitors.

15. (canceled)

16. (canceled)

17. The method of claim 2, wherein the defined level is 1.2 times, 1.5 times, 2.0 times, 2.5 times, 3.0 times, 3.5 times, 4.0 times, 5.0 times, or 6.0 times expression level of HIF-1α in parental cells.

18. The method of claim 17, wherein the phosphaplatin compound has a structure of formula I or II: 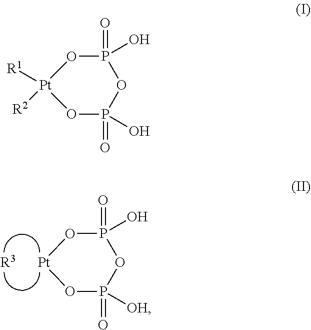
or a pharmaceutically acceptable salt thereof, wherein R¹ and R² are each independently selected from methyl amine, ethyl amine, propyl amine, isopropyl amine, butyl amine, cyclohexane amine, aniline, pyridine, and substituted pyridine; and R³ is selected from 1,2-ethylenediamine and cyclohexane-1,2-diamine.

19. The method of claim 17, wherein the phosphaplatin compound is selected from: 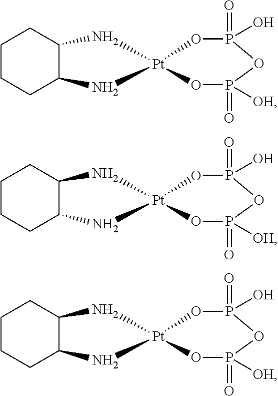
or pharmaceutically acceptable salts, and mixtures thereof.

20. The method of claim 17, the phosphaplatin compound is (R,R)-1,2-cyclohexanediamine-(pyrophosphato)platinum(II) (or “PT-112”), or a pharmaceutically acceptable salt thereof.

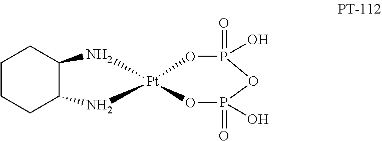

21. The method of claim 17, wherein the cancer or tumor is selected from the group consisting of gynecological cancers, genitourinary cancers, lung cancers, head-and-neck cancers, skin cancers, gastrointestinal cancers, breast cancers, bone and chondroital cancers, soft tissue sarcomas, thymic epithelial tumors, and hematological cancers.

22. The method of claim 5, wherein the phosphaplatin compound has a structure of formula I or II. 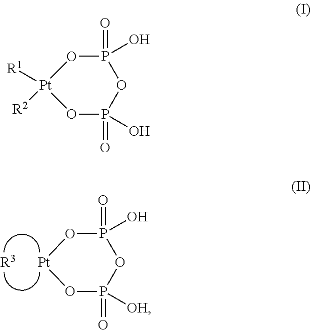
or a pharmaceutically acceptable salt thereof, wherein R¹ and R² are each independently selected from methyl amine, ethyl amine, propyl amine, isopropyl amine, butyl amine, cyclohexane amine, aniline, pyridine, and substituted pyridine; and R³ is selected from 1,2-ethylenediamine and cyclohexane-1,2-diamine.
